(19) United States
(12) Patent Application Publication
(10) Pub. No.: US 2013/0302415 A1
(43) Pub. Date: Nov. 14, 2013

(54) Pharmaceutical Composition

(71) Applicant: CIPLA Limited, Mumbai (IN)

(72) Inventors: Amar Lulla, Mumbai (IN); Geena Malhotra, Mumbai (IN)

(21) Appl. No.: 13/941,525

(22) Filed: Jul. 14, 2013

Related U.S. Application Data
(63) Continuation of application No. 13/641,852.

(30) Foreign Application Priority Data
Apr. 20, 2010 (IN) 1296/MUM/2010

(51) Int. Cl. A61K 9/14; A61K 31/536

(52) U.S. Cl. CPC A61K 9/14; A61K 31/536
USPC 424/451; 428/402; 424/489; 514/230.5; 424/464

(57) ABSTRACT

A pharmaceutical composition comprising efavirenz wherein the efavirenz is in the form of nanoparticles is disclosed.

### CROSS-REFERENCE TO RELATED CASES

This application is filed under 35 U.S.C. §111(a) as a continuation application which claims priority under 35 U.S.C. §119, 35 U.S.C. §120, and the Patent Cooperation Treaty to: parent application U.S. Ser. No. 13/641,852 filed under 35 U.S.C. §371 on Oct. 17, 2012; which claims priority to PCT/GB2011/000620 filed under the authority of the Patent Cooperation Treaty on Apr. 20, 2011, published; which claims priority to Indian Application Ser. No. 1296/MUM/2010 filed Apr. 20, 2010.

### FIELD OF INVENTION

The present invention relates to a pharmaceutical composition comprising an antiretroviral drug, a process for preparing such composition, therapeutic uses and method of 5 treatment employing the same.

### BACKGROUND & PRIOR ART

Efavirenz is the international non-proprietary name for non-nucleoside reverse transcriptase inhibitor (S)-6-chloro-4-cyclopropylethynyl-4-trifluoromethyl-1,4-dihydro-2H-3,1-benzoxazin-2-one belonging to class of benzoxazinones. Efavirenz has the following structural formula:

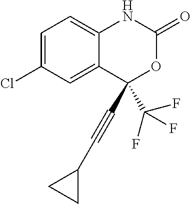 [SMILES: O=C1Nc2ccc(Cl)cc2[C@@](C#CC2CC2)(C(F)(F)F)O1]

Efavirenz is effective in the treatment of the human immunodeficiency virus (HIV) which is the retrovirus that causes progressive destruction of the human immune system resulting in onset of AIDS. Efavirenz is a highly potent reverse transcriptase inhibitor and is effective against HIV reverse transcriptase resistance. It is a crystalline lipophilic solid with a log octanol water partition coefficient of 5.4 and an aqueous solubility of 9.0 μg/ml.

Efavirenz is classified in class II drugs (low solubility, high permeability) of the Biopharmaceutical Classification System. Class II drugs like efavirenz demonstrate poor gastrointestinal (GI) absorption due to inadequate drug solubility in GI fluids. Furthermore, efavirenz is a crystalline lipophilic solid with an aqueous solubility of 9.0 μg/mL and with a low intrinsic dissolution rate (IDR) of 0.037 mg/cm 2/min. The drugs with less than 0.1 mg/cm 2/min of IDR have dissolution as a rate-limiting step in absorption, which is further affected by the fed/fasted state of the patient. This in turn can affect the peak plasma concentration, making calculation of dosage and dosing regimens more complex.

This suggests the importance of dissolution improvement for efavirenz. Moreover, most of these new chemical entities despite their high permeability, are only absorbed in the upper small intestine. Consequently, if these drugs are not completely released in gastro intestinal tract area, they have low bioavailability. Thus, there is a need to increase the therapeutic dose of the drug in order to obviate this disadvantage; however increasing the dose may lead to increase in the side effects of the drug.

Various prior art formulations have been reported to improve the solubility of the efavirenz in the GI tract. For example, one of the approaches used is encapsulation of drug in cyclodextrins using 1:1 molar ratio as reported by Indrajit et al in Macromolecular symposia in 2010, 287, 51-59. However considering the high dose of efavirenz, it is practically difficult to develop oral dosage form using cyclodextrins.

Solid dispersion and PEGylation techniques have also been proposed by Madhavi et al in “Dissolution enhancement of efavirenz by solid dispersion and PEGylation techniques”; International Journal of Pharmaceutical Investigation, 2011 (1), 29-34, wherein the drug and the carrier are added to a common solvent followed by homogenization and evaporation of the solvent to form solid dispersion of efavirenz. However recrystallization of amorphous solid dispersions due to temperature, humidity, and the amount of polymer may lead to a reduction in the dissolution rate, and consequently reduce bioavailability. Further the article also states that drug-PEG conjugates in 1:1 and 1:2 w/w ratios were prepared by dissolving efavirenz and PEG 6000 separately in organic solvent and then pouring the solution of the drug into the solution of PEG while stirring, incubating the mixture overnight and then evaporating the solvent to yield the PEGylated compound. However PEGylation is a complex procedure requiring many processing steps.

WO99/61026 discloses a tablet dosage form of efavirenz wherein lactose is added extragranularly to obtain a stable tablet formulation which is bioequivalent to the capsule formulation of efavirenz. However, the patent does not provide any bioequivalence data.

U.S. Pat. No. 6,555,133 B2 provides improved oral dosage forms of efavirenz containing one or more super disintegrants that enhance the dissolution rate of the drug in the gastrointestinal tract thereby improving the rate and extent of absorption of drug in the body. However use of higher amount of a super disintegrant like sodium starch glycolate may lead to a negative effect on the disintegration of the tablets due to formation of a viscous gel layer formed by sodium starch glycolate that may form a thick barrier to the further penetration of the disintegration medium and hinder the disintegration of tablets [Development of Fast Dispersible Aceclofenac Tablets: Effect of Functionality of Superdisintegrant, C. Mallikarjuna Setty and et al; Received Feb. 7, 2007; Revised Jan. 16, 2008; Accepted Mar. 12, 2008]

Therefore, the improvement of efavirenz solubility thereby its oral bio-availability while reducing the dose of drug remains one of most challenging aspects especially for oral drug delivery system. It is desirable to provide compositions of efavirenz exhibiting enhanced bioavailability compared to the prior art formulations. Thus there still exists an unmet need to develop an efavirenz formulation with improved solubility and dissolution properties of the drug.

### OBJECT OF THE INVENTION

The object of the present invention is to provide a pharmaceutical composition of efavirenz having improved solubility and dissolution.

Another object of the present invention is to provide a method of manufacturing a pharmaceutical composition comprising efavirenz.

### SUMMARY OF THE INVENTION

According to one aspect of the invention there is provided a composition comprising efavirenz in the form of particles, wherein substantially all the particles have a particle size less than or equal to 1 micrometre.

In a preferred embodiment, the composition further comprises at least one surface stabilizer, at least one viscosity building agent and at least one polymer, wherein substantially all the particles have a particle size less than or equal to 1 micrometre.

In a preferred embodiment, all the particles have a particle size above 1 nanometre.

The composition described above may comprise a pharmaceutical composition, or may be used to form a pharmaceutical composition.

According to another aspect of the present invention there is provided a pharmaceutical composition comprising efavirenz or a pharmaceutically acceptable salt, solvate, derivative, hydrate, polymorph, or mixtures thereof wherein the particle size of efavirenz is in nanometre range.

According to yet another aspect of the present invention there is provided a process for preparing a pharmaceutical composition comprising efavirenz or a pharmaceutically acceptable salt, solvate, derivative, hydrate, polymorph, or mixtures thereof wherein the particle size of efavirenz is in nanometre range.

According to another aspect of the present invention there is provided a method of treatment using a pharmaceutical composition according to present invention.

### DETAILED DESCRIPTION

Efavirenz is a class II drug having low solubility and low dissolution. Bioavailability is the degree to which a drug becomes available to the target tissue after administration. Many factors can affect bioavailability including the dosage form, particle size, various properties, e.g., dissolution rate of the drug. Poor bioavailability is a significant problem encountered in the development of pharmaceutical compositions, particularly for those containing an active ingredient that is poorly soluble in water. Poorly water soluble drugs, i.e., those having a solubility less than about 10 mg/ml, tend to be eliminated from the gastrointestinal tract before being absorbed into the circulation. Therefore development of efavirenz formulations poses a challenge to an inventor. The inventors of the present invention have surprisingly found that the dissolution property of efavirenz was greatly improved by reducing the particle size of efavirenz to nanometre range thus leading to better absorption and bioavailability of the drug from the GI tract.

The present invention thus provides a pharmaceutical composition comprising efavirenz in nano form and a process for preparing the same. The term “efavirenz” as used herein in the entire specification and claims is employed in a broad sense to include not only efavirenz but its pharmaceutically acceptable salts, solvates, derivatives, prodrugs, racemic mixtures, polymorphs thereof.

Nanonization of hydrophobic drugs generally involves the production of drug nanocrystals through either chemical precipitation [bottom-up technology] or disintegration [top-down technology]. Different methods may be utilized to reduce the particle size of the hydrophobic drugs for ex: Huabing Chen and et al, discusses the various methods to develop nanoformulations in “Nanonization strategies for poorly water-soluble drugs,” Drug Discovery Today, Volume 00, Number 00, March 2010.

The nanoparticles of the present invention may be obtained by any of the process such as but not limited to milling, precipitation and homogenization.

According to one embodiment of the present invention, the process of milling comprises dispersing efavirenz particles in a liquid dispersion medium in which efavirenz is poorly soluble, followed by applying mechanical means in the presence of grinding media like milling pearls to reduce the particle size of efavirenz to the desired average particle size.

According to another embodiment of the present invention, the process of precipitation involves the formation of crystalline or semi-crystalline efavirenz nanoparticles by nucleation and the growth of drug crystals. In a typical procedure, drug molecules are first dissolved in an appropriate organic solvent such as acetone, tetrahydrofuran or N-methyl-2-pyrrolidone at a super saturation concentration to allow for the nucleation of drug seeds. Drug nanocrystals are then formed by adding the organic mixture to an antisolvent like water in the presence of stabilizers such as Tween 80, Poloxamer 188 or lecithin. The choice of solvents and stabilizers and the mixing process are key factors to control the size and stability of the drug nanocrystals.

According to one another embodiment of the present invention, the process of homogenization involves passing a suspension of crystalline efavirenz and stabilizers through the narrow gap of a homogenizer at high pressure (500-2000 bar). The pressure creates powerful disruptive forces such as cavitation, collision and shearing, which disintegrate coarse particles to nanoparticles.

According to one more embodiment of the present invention, the process of spray-freeze drying involves the atomization of an aqueous efavirenz solution into a spray chamber filled with a cryogenic liquid (liquid nitrogen) or halocarbon refrigerant such as chlorofluorocarbon or fluorocarbon. The water is removed by sublimation after the liquid droplets solidify.

According to a still another embodiment of the present invention, the process of supercritical fluid technology involves controlled crystallization of efavirenz from dispersion in supercritical fluids, carbon dioxide.

According to another embodiment of the present invention, the process of double emulsion/solvent evaporation technique involves preparation of oil/water (o/w) emulsions with subsequent removal of the oil phase through evaporation. The emulsions are prepared by emulsifying the organic phase containing efavirenz, polymer and organic solvent in an aqueous solution containing emulsifier. The organic solvent diffuses out of the polymer phase into the aqueous phase, and is then evaporated, forming efavirenz-loaded polymeric nanoparticles.

According to a further embodiment of the present invention, the process of PRINT (Particle replication in non-wetting templates) involves utilization of a low surface energy fluoropolymeric mold that enables high-resolution imprint lithography, to fabricate a variety of organic particles. PRINT can precisely manipulate particle size of efavirenz ranging from 20 nm to more than 100 nm.

According to one further embodiment of the present invention, the process of thermal condensation involves use of capillary aerosol generator (CAG) to produce high concentration condensation submicron to nano sized aerosols from efavirenz solutions.

According to still further embodiment of the present invention, the process of ultrasonication involves application of ultrasound during particle synthesis or precipitation, which leads to smaller particles of efavirenz and increased size uniformity.

According to another embodiment of the present invention, the process of spray drying involves supplying the feed solution at room temperature and pumping it through the nozzle where it is atomized by the nozzle gas. The atomized solution is then dried by preheated drying gas in a special chamber to remove water moisture from the system, thus forming dry particles of efavirenz.

According to a preferred embodiment of the present invention, the nanonization of efavirenz involves nanomilling efavirenz with at least one surface stabilizer, at least one viscosity building agent and at least one polymer. The nanomilled efavirenz according to present invention exhibits a particle size of less than or equal to 5 μm, preferably less than or equal to 3 μm, more preferably less than or equal to 1 μm. The present invention thus provides a pharmaceutical composition comprising granules of nanomilled efavirenz wherein the granules comprise at least one surface stabilizer, at least one viscosity building agent and at least one polymer along with efavirenz and optionally other pharmaceutically acceptable carriers.

The expression surface stabilizer according to the present inventions means a surfactant that is capable of stabilizing the increased surfaced charge of the nanomilled drug. Any surfactant is suitable, whether it may be amphoteric, non-ionic, cationic or anionic. Suitable surfactants may be included in the solid dosage form as provided by the present invention. Non-limiting examples from anionic, cationic, non-ionic and amphoteric groups include Polysorbates, Sodium dodecyl sulfate (sodium lauryl sulfate), Lauryl dimethyl amine oxide, Docusate sodium, Cety trimethyl ammonium bromide (CTAB) Polyethoxylated alcohols, Polyoxyethylene sorbitan, Octoxynol, N,N-dimethyldodecylamine-N-oxide, Hexadecyltrimethylammonium bromide, Polyoxyl 10 lauryl ether, Brij, Bile salts (sodium deoxycholate, sodium cholate), Polyoxyl castor oil, Nonylphenol ethoxylate, Cyclodextrins, Lecithin, Methylbenzethonium chloride.

Carboxylates, Sulphonates, Petroleum sulphonates, alkylbenzenesulphonates, Naphthalenesulphonates, Olefin sulphonates, Alkyl sulphates, Sulphates, Sulphated natural oils & fats, Sulphated esters, Sulphated alkanolamides, Alkylphenols, ethoxylated & sulphated, Ethoxylated aliphatic alcohol, polyoxyethylene surfactants, carboxylic esters

Polyethylene glycol esters, Anhydrosorbitol ester & it's ethoxylated derivatives, Glycol esters of fatty acids, Carboxylic amides, Monoalkanolamine condensates, Polyoxyethylene fatty acid amides, Quaternary ammonium salts, Amines with amide linkages, Polyoxyethylene alkyl & alicyclic amines, N,N,N,N tetrakis substituted ethylenediamines 2alkyl 1-hydroxyethyl 2-imidazolines, N-coco 3-aminopropionic acid/sodium salt N-tallow3-iminodipropionate disodium salt, N-carboxymethyl n dimethyl n-9 octadecenyl ammonium hydroxide, n-cocoamidethyl n-hydroxyethylglycine sodium salt etc.

The term viscosity builder means excipients that are capable of stabilizing the nanoparticles by increasing the viscosity of the formulation and thus preventing physical interaction of nanoparticles under the operating conditions employed. Examples of such excipients are derivatives of sugars, such as lactose, sucrose, saccharose, hydrolyzed starch (maltodextrin) and the like. Mixtures are also suitable.

Suitable examples of polymers include but are not limited to cellulose derivates like hydroxypropylcellulose, hydroxymethylcellulose, hydroxypropylmethylcellulose, methylcellulose polymers hydroxyethylcellulose, sodium carboxymethylcellulose, carboxymethylene and carboxymethyl hydroxyethylcellulose; acrylics like acrylic acid, acrylamide, and maleic anhydride polymers and copolymers. Polymer blends are also suitable.

The present invention provides a process of preparing a pharmaceutical composition, which process comprises the steps of: homogenizing a drug, at least one surface active agent, at least one viscosity building agent, at least one polymer to produce a homogenized dispersion of the drug in the surface active agent, the viscosity building agent and the polymer; nanomilling the homogenized dispersion of step one to produce a nanomilled slurry; adsorbing the nanomilled slurry on a carrier to form granules.

In one embodiment the percentage weight of active ingredient in the slurry ranges from 5% to 60% w/w.

The granules may either be encapsulated in capsules or be compressed to form tablets or may 5 be provided as sachets or be provided as powders for reconstitution.

The solid dosage form according to the present invention may optionally be coated. More preferably, the formulation may be seal coated and further film coated.

Alternatively the nanomilled slurry may be used to formulate liquid dosage forms like suspension.

The term carrier used herein includes one more of pharmaceutically acceptable ingredients but not limited to carriers, diluents or fillers, binders, lubricants, glidants and disintegrants. Non-limiting examples of suitable pharmaceutically acceptable carriers, diluents or fillers for use in the solid dosage form as provided by the present invention include lactose (for example, spray-dried lactose, a-lactose, (3-lactose), lactose available under the trade mark Tablettose, various grades of lactose available under the trade mark Pharmatose or other commercially available forms of lactose, lactitol, saccharose, sorbitol, mannitol, dextrates, dextrins, dextrose, maltodextrin, croscarmellose sodium, microcrystalline cellulose (for example, microcrystalline cellulose available under the trade mark Avicel), hydroxypropylcellulose, L-hydroxypropylcellulose (low substituted), hydroxypropyl methylcellulose (HPMC), methylcellulose polymers (such as, for example, Methocel A, Methocel A4C, Methocel A15C, Methocel A4M), hydroxyethylcellulose, sodium carboxymethylcellulose, carboxymethylene, carboxymethyl hydroxyethylcellulose and other cellulose derivatives, starches or modified starches (including potato starch, corn starch, maize starch and rice starch) and the like.

Typically glidants and lubricants may also be included in the solid dosage form as provided by the present invention. Non-limiting examples include stearic acid and pharmaceutically acceptable salts or esters thereof (for example, magnesium stearate, calcium stearate, sodium stearyl fumarate or other metallic stearate), talc, waxes (for example, microcrystalline waxes) and glycerides, light mineral oil, PEG, silica acid or a derivative or salt thereof (for example, silicates, silicon dioxide, colloidal silicon dioxide and polymers thereof, crospovidone, magnesium aluminosilicate and/or magnesium alumino metasilicate), sucrose ester of fatty acids, hydrogenated vegetable oils (for example, hydrogenated castor oil), or mixtures thereof or any other suitable lubricant.

Suitably one or more binders are also present in the solid dosage form as provided by the present invention and non-limiting examples of suitable binders are, for example, polyvinyl pyrrolidone (also known as povidone), polyethylene glycol(s), acacia, alginic acid, agar, calcium carrageenan, cellulose derivatives such as ethyl cellulose, methyl cellulose, hydroxypropyl cellulose, hydroxypropyl methyl cellulose, sodium carboxymethylcellulose, dextrin, gelatin, gum arabic, guar gum, tragacanth, sodium alginate, or mixtures thereof or any other suitable binder.

Suitable disintegrants may also be present in the formulation according to the present invention, which includes, but are not limited to hydroxylpropyl cellulose (HPC), low density HPC, carboxymethylcellulose (CMC), sodium CMC, calcium CMC, croscarmellose sodium; starches exemplified under examples of fillers and also carboxymethyl starch, hydroxylpropyl starch, modified starch; crystalline cellulose, sodium starch glycolate; alginic acid or a salt thereof, such as sodium alginate or their equivalents and any combination thereof.

In one embodiment of the present invention there is provided a process of preparing a pharmaceutical composition according to the present invention which process comprises the step of 1. Homogenizing the dispersion of Efavirenz, docusate sodium, sucrose, HPMC 2. Nanomilling the homogenized dispersion of step one 3. Adsorbing the nanomilled slurry of step 2 on a mixture of lactose monohydrate, microcrystalline cellulose and crospovidone to form granules.

In yet another preferred embodiment of the present invention there is provided a process of preparing a pharmaceutical composition, which process comprises: (1). preparing a dispersion of Efavirenz with Docusate sodium, HPMC, sodium lauryl sulphate and sucrose in purified water under stifling conditions (2). Homogenizing the step (1) dispersion and then Nanomilling the homogenized dispersion (3) Adsorbing the nanomilled drug by spraying the nanomilled slurry on lactose monohydrate, microcrystalline cellulose and crospovidone mixture in fluidized bed granulator. (4) Drying and blending the granules obtained. (5) Lubricating the granules and finally compressing into tablets (6) the tablets obtained were seal coated and then film coated.

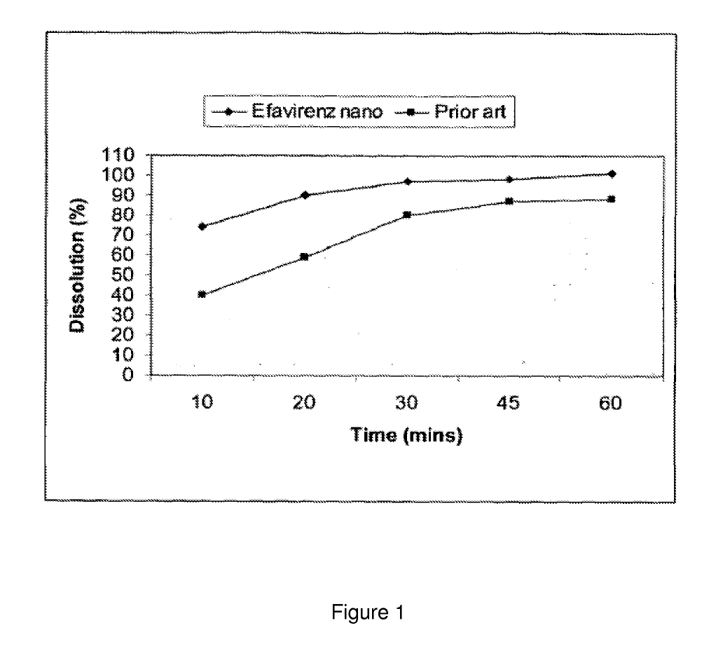

The nanomilled efavirenz composition prepared according to the present invention exhibited a dissolution profile which is showing an improvement over the prior art composition as evident from FIG. 1. This might further lead to a considerably enhanced bioavailability of the active ingredient compared to that obtained with the compositions of the prior art. Further as may be noted from the dissolution data, a suitable dose of efavirenz that may be administered according to the present invention may be in the range of about 300 mg to about 600 mg, which may lead to reduced side effects of the active ingredient.

There is further provided by the present invention a solid dosage form substantially as hereinbefore described, for use in treating disorders or conditions that respond to, or are prevented, ameliorated or eliminated by, the administration of efavirenz. More preferably, there is further provided by the present invention a solid dosage form substantially as hereinbefore described, for use in the treatment of Human immunodeficiency virus [HIV]. Efavirenz is also used in combination with other antiretroviral agents as part of an expanded post exposure prophylaxis regimen to reduce the risk of HW infection in people exposed to a significant risk

It can be appreciated from the above mentioned method of treatment description in accordance with the present invention that it can be beneficial to provide, recommend or label a solid dosage form according to the present invention for administration with one or more other therapeutically active compounds used for treatment of HIV infection.

The present invention is further explained with the following non-limiting examples and with the aid of dissolution profile of Efavirenz tablets prepared according to present invention with innovators tablets.

The following example is for the purpose of illustration of the invention only and is not intended in any way to limit the scope of the present invention.

### Example 1

### Formula

| Sr. No. | Ingredients | Qty mg/tablet |
|---|---|---|
| 1. | Efavirenz IP | 600.00 |
| 2. | Docusate Sodium IP | 06.00 |
| 3. | Hydroxypropylmethylcellulose 3 cps IP | 50.00 |
| 4. | Sodium lauryl sulphate IP | 16.55 |
| 5. | Sucrose IP | 100.00 |
| 6. | Purified water IP | q.s |
| 7. | Lactose Monohydrate(200 mesh) IP | 325.00 |
| 8. | Microcrystalline Cellulose IP (Avicel PH 101) | 320.56 |
| 9. | Crospovidone IP | 50.00 |
| 10. | Crospovidone IP | 36.89 |
| 11. | Magnesium Stearate IP | 08.00 |
| | Total | 1513.00 |
| 12. | Hydroxypropylmethylcellulose 3 cps IP | 15.00 |
| 13. | Isopropyl Alcohol IP | q.s |
| 14. | Dichloromethane BP | q.s |
| | Total | 1528.00 |
| V] | Film Coating | |
| 15. | Opadry AMB White OY-B-28920 INH | 45.00 |
| 16. | Purified Water IP | q.s |
| | Total | 1573.00 |

### Process:

1. Dispersion of efavirenz with Docusate sodium, HPMC, sodium lauryl sulphate and sucrose was prepared in purified water under stifling conditions.
2. Above dispersion was homogenized and then nanomilled.
3. Nanomilled drug slurry was adsorbed by spraying on lactose monohydrate, microcrystalline cellulose and crospovidone mixture in a fluidized bed granulator.
4. Granules obtained were sized and lubricated.
5. Lubricated granules were finally compressed into tablets.
6. The tablets obtained were seal coated and then film coated.

### Example 2

### Formula

| Sr. No. | Ingredients | Qty mg/tablet |
|---|---|---|
| 1. | Efavirenz IP | 300.00 |
| 2. | Docusate Sodium IP | 03.00 |
| 3. | Hydroxypropylmethylcellulose 3 cps IP | 25.00 |
| 4. | Sodium lauryl sulphate IP | 8.27 |
| 5. | Sucrose IP | 50.00 |
| 6. | Purified water IP | q.s |
| 7. | Lactose Monohydrate(200 mesh) IP | 162.5 |
| 8. | Microcrystalline Cellulose IP (Avicel PH 101) | 160.28 |
| 9. | Crospovidone IP | 25.00 |
| 10. | Crospovidone lP | 18.44 |
| 11. | Magnesium Stearate IP | 04.00 |
| | Total | 756.00 |
| 12. | Hydroxypropylmethylcellulose 3 cps IP | 15.00 |
| 13. | Isopropyl Alcohol IP | q.s |
| 14. | Dichloromethane BP | q.s |
| | Total | 771.00 |
| V] | Film Coating | |
| 15. | Opadry AMB White OY-B-28920 INH | 22.5 |
| 16. | Purified Water IP | q.s |
| | Total | 793.50 |

### Process:

1. Dispersion of efavirenz with Docusate sodium, HPMC, sodium lauryl sulphate and sucrose was prepared in purified water under stirring conditions.
2. Above dispersion was homogenized and then nanomilled.
3. Nanomilled drug slurry was adsorbed by spraying on lactose monohydrate, microcrystalline cellulose and crospovidone mixture in a fluidized bed granulator.
4. Granules obtained were sized and lubricated.
5. Lubricated granules were finally compressed into tablets.
6. The tablets obtained were seal coated and then film coated.

### Example: 3

### Dissolution of a Composition According to the Invention and a Composition According to the Prior Art

According to the present invention a dissolution study was carried out in an aqueous medium containing a surfactant, 2% SLS. The paddle method (US Pharmacopoeia) was used under the following conditions: volume of medium1000 ml; medium temperature: 3T C.; blade rotation speed 50 rpm; samples taken: every 10 minutes.

TABLE 1

% dissolved

| Interval | Efavirenz tablets | Prior art tablets |
|---|---|---|
| (mins) | 300 mg | 600 mg |
| 10 | 74 | 40 |
| 20 | 90 | 59 |
| 30 | 97 | 80 |
| 45 | 98 | 87 |
| 60 | 101 | 88 |

The composition according to present invention consisted of Efavirenz 300 mg tablets prepared according to Example 2. The prior art composition contained Efavirenz [600 mg] croscarmellose sodium, hydroxypropyl cellulose, lactose monohydrate, magnesium stearate, microcrystalline cellulose, and sodium lauryl sulfate.

The results obtained are shown graphically in FIG. 1, on which the percentage of dissolution is shown. As shown in table 1 and FIG. 1, approximately 75% of the active from nano composition dissolved in 10 minutes and almost 100% of active dissolved within an hour while prior art formulation dissolved only 88% in one hour. These results clearly show that the compositions of the present invention have a dissolution profile which is distinctly better than the prior art composition.

It will be readily apparent to one skilled in the art that varying substitutions and modifications may be made to the invention disclosed herein without departing from the spirit of the invention. Thus, it should be understood that although the present invention has been specifically disclosed by the preferred embodiments and optional features, modification and variation of the concepts herein disclosed may be resorted to by those skilled in the art, and such modifications and variations are considered to be falling within the scope of the invention.

It is to be understood that the phraseology and terminology used herein is for the purpose of description and should not be regarded as limiting. The use of “including,” “comprising,” or “having” and variations thereof herein is meant to encompass the items listed thereafter and equivalents thereof as well as additional items.

It must be noted that, as used in this specification and the appended claims, the singular forms a,” “an” and “the” include plural references unless the context clearly dictates otherwise.

What is claimed is:

1. A composition comprising efavirenz in the form of particles, wherein substantially all the particles have a particle size less than or equal to 1 micrometre.

2. A composition according to claim 1, further comprising at least one surface stabilizer, at least one viscosity building agent and at least one polymer, wherein substantially all the particles have a particle size less than or equal to 1 micrometre.

3. A composition according to claim 2, wherein the surface stabilizer is a surfactant.

4. A composition according to claim 3, wherein the surfactant is an amphoteric, non-ionic, cationic or anionic surfactant.

5. A composition according to claim 3, wherein the surfactant is a polysorbate; sodium dodecyl sulfate (sodium lauryl sulfate); lauryl dimethyl amine oxide; docusate sodium; cetyl trimethyl ammonium bromide (CTAB); a polyethoxylated alcohol; a polyoxyethylene sorbitan; Octoxynol; N,N-dimethyldodecylamine-N-oxide; hexadecyl trimethylammonium bromide, polyoxyl lauryl ether, brij, a bile salt, sodium deoxycholate, sodium cholate; a polyoxyl castor oil; nonylphenol ethoxylate; a Cyclodextrin; lecithin; methylbenzethonium chloride; a carboxylate; a sulphonate; a petroleum sulphonate; an alkylbenzenesulphonate; a naphthalenesulphonate; an olefin sulphonate; a sulphate surfactant; an alkyl sulphate; a sulphated natural oil or fat; a sulphated ester; a sulphated alkanolamide; an alkylphenol, optionally ethoxylated and sulphated; an ethoxylated aliphatic alcohol; polyoxyethylene; a carboxylic ester; a polyethylene glycol ester; an anhydrosorbitol ester or an ethoxylated derivative thereof, a glycol ester of a fatty acid; a carboxylic amide; a monoalkanolamine condensate; a polyoxyethylene fatty acid amide; a quaternary ammonium salt; an amine with amide linkages; a polyoxyethylene alkyl amine; a polyoxyethylene alicyclic amine; a N,N,N,N tetrakis substituted ethylenediamine; a 2-alkyl-1-hydroxyethyl-2-imidazoline; N-coco-3-aminopropionic acid or a sodium salt thereof; N-tallow-3-iminodipropionate disodium salt; N-carboxymethyl-n-dimethyl-n-9 octadecenyl ammonium hydroxide; n-cocoamidethyl-n-hydroxyethylglycine sodium salt; or mixtures thereof.

6. A composition according to claim 3, wherein the surfactant is docusate sodium and/or sodium lauryl sulphate.

7. A composition according to claim 2, wherein the viscosity building agent is lactose; sucrose; saccharose; a hydrolyzed starch, such as maltodextrin; or mixtures thereof.

8. A composition according to claim 7, wherein the viscosity building agent is sucrose.

9. A composition according to claim 2, wherein the polymer is hydroxypropylcellulose; hydroxymethylcellulose; hydroxypropylmethylcellulose; a methylcellulose polymer; hydroxyethylcellulose; sodium carboxymethylcellulose; carboxymethylene hydroxyethylcellulose and/or carboxymethyl hydroxyethylcellulose; an acrylic polymer, acrylic acid, acrylamide, and maleic anhydride polymers and copolymers; or a blend thereof; or mixtures thereof.

10. A composition according to claim 9, wherein the polymer is hydroxypropylmethylcellulose.

11. A composition according to claim 1, wherein substantially all the particles have a particle size above 1 nanometre.

12. A composition according to claim 1, further comprising a pharmaceutically acceptable carrier, wherein said particles have been adsorbed onto the surface of the carrier.

13. A pharmaceutical composition comprising a composition according to claim 12.

14. A pharmaceutical composition according to claim 13, wherein the carrier comprises: one or more diluents or fillers; one or more binders; one or more lubricants; one or more glidants; one or more disintegrants; or a mixture thereof.

15. A pharmaceutical composition according to claim 13, wherein the carrier comprises lactose monohydrate, microcrystalline cellulose and crospovidone or mixtures thereof.

16. A pharmaceutical composition according to claim 13, which is in the form of a tablet dosage form, a powder dosage form, a capsule dosage form or a liquid dosage form.

17. A process for preparing a pharmaceutical composition, which process comprises the steps of: homogenizing efavirenz, at least one surface stabiliser, at least one viscosity building agent, and at least one polymer to produce a homogenized dispersion of the efavirenz in the surface active agent, the viscosity building agent and the polymer; milling said homogenized dispersion to produce a slurry of particles having a particle size less than or equal to 1 micrometre; and adsorbing the milled slurry on a carrier to form granules.

18. A process according to claim 17, wherein the granules are compressed to form tablets, or are encapsulated in capsules, or are provided as a powder dosage form.

19. A process according to claim 17, wherein the granules are used to form a liquid dosage formulation.

20. A process according to claim 17, wherein the surface stabilizer is a surfactant.

21. A process according to claim 20, wherein the surfactant is an amphoteric, non-ionic, cationic or anionic surfactant.

22. A process according to claim 20, wherein the surfactant is a polysorbate; sodium dodecyl sulfate (sodium lauryl sulfate); lauryl dimethyl amine oxide; docusate sodium; cetyl trimethyl ammonium bromide (CTAB); a polyethoxylated alcohol; a polyoxyethylene sorbitan; Octoxynol; N,N-dimethyldodecylamine-N-oxide; hexadecyl trimethylammonium bromide, polyoxyl 10 lauryl ether, brij, a bile salt, sodium deoxycholate, sodium cholate; a polyoxyl castor oil; nonylphenol ethoxylate; a Cyclodextrin; lecithin; methylbenzethonium chloride; a carboxylate; a sulphonate; a petroleum sulphonate; an alkylbenzenesulphonate; a naphthalenesulphonate; an olefin sulphonate; a sulphate surfactant; an alkyl sulphate; a sulphated natural oil or fat; a sulphated ester; a sulphated alkanolamide; an alkylphenol, optionally ethoxylated and sulphated; an ethoxylated aliphatic alcohol; polyoxyethylene; a carboxylic ester; a polyethylene glycol ester; an anhydrosorbitol ester or an ethoxylated derivative thereof; a glycol ester of a fatty acid; a carboxylic amide; a monoalkanolamine condensate; a polyoxyethylene fatty acid amide; a quaternary ammonium salt; an amine with amide linkages; a polyoxyethylene alkyl amine; a polyoxyethylene alicyclic amine; a N,N,N,N tetrakis substituted ethylenediamine; a 2-alkyl-1-hydroxyethyl-2-imidazoline; N-coco-3-aminopropionic acid or a sodium salt thereof; N-tallow-3-iminodipropionate disodium salt; N-carboxymethyl-n-dimethyl-n-9 octadecenyl ammonium hydroxide; n-cocoamidethyl-n-hydroxyethylglycine sodium salt; or mixtures thereof.

23. A process according to claim 20, wherein the surfactant is docusyl sodium 20 and/or sodium lauryl sulphate.

24. A process according to claim 17, wherein the viscosity building agent is lactose; sucrose; saccharose; a hydrolyzed starch, such as maltodextrin; or a mixture thereof.

25. A process according to claim 24, wherein the viscosity building agent is sucrose.

26. A process according to claim 17 wherein the polymer is hydroxypropylcellulose; hydroxymethylcellulose; hydroxypropylmethylcellulose; a methylcellulose polymer; hydroxyethylcellulose; sodium carboxymethylcellulose; carboxymethylene hydroxyethylcellulose and/or carboxymethyl hydroxyethylcellulose; an acrylic polymer, acrylic acid, acrylamide, and maleic anhydride polymers and copolymers; or a blend thereof, or a mixture thereof.

27. A process according to claim 26, wherein the polymer is hydroxypropylmethylcellulose.

28. A process according to claim 1, wherein substantially all the particles have a particle size above 1 nanometre.

29. A process according to claim 17 wherein the carrier comprises: one or more diluents or fillers; one or more binders; one or more lubricants; one or more glidants; 10 one or more disintegrants; or a mixture thereof.

30. A process according to claim 17, wherein the carrier comprises lactose monohydrate, microcrystalline cellulose and crospovidone or mixtures thereof.

31. A process according to claim 17, wherein the milled slurry is adsorbed onto the particles by spraying the slurry onto the granules in a fluidized bed granulator.

32. A process according claim 17, further comprising drying and blending the granules after the step of adsorbing the milled slurry.

33. A composition comprising efavirenz in the form of particles, wherein substantially all the particles have a particle size less than or equal to 1 micrometre.

34. The composition of claim 33 comprising efavirenz in the form of particles, wherein substantially all the particles have a particle size above 1 nanometre.

35. A method of treating HIV comprising administering a therapeutically effective amount of a composition comprising efavirenz in the form of particles, wherein substantially all the particles have a particle size less than or equal to 1 micrometre.
